(19) United States
(12) Patent Application Publication
(10) Pub. No.: US 2024/0299439 A1
(43) Pub. Date: Sep. 12, 2024

(54) TNF-alpha Protein Inhibiting mRNA Vaccine

(71) Applicants: Sarfaraz K. Niazi, Deerfield, IL (US); Matthias Magoola, Kampala (UG); Zafeer Ahmad, Wadsworth, IL (US)

(72) Inventors: Sarfaraz K. Niazi, Deerfield, IL (US); Matthias Magoola, Kampala (UG); Zafeer Ahmad, Wadsworth, IL (US)

(73) Assignee: RNA Therapeutics, Inc., Deerfield, IL (US)

(21) Appl. No.: 18/406,166

(22) Filed: Jan. 7, 2024

(51) Int. Cl. A61K 31/7105; A61K 9/51; A61K 39/00

(52) U.S. Cl. CPC A61K 31/7105; A61K 9/5123; A61K 2039/53; A61K 2039/55555; C12N 2830/50

(57) ABSTRACT

TNF-alpha (Tumor Necrosis Factor-alpha) is a cytokine, a protein involved in the inflammation process in the body. It plays a crucial role in the immune system, primarily produced by activated macrophages the white blood cells. TNF-alpha is critical in the inflammatory response to infections and diseases, but it can also contribute to inflammatory and autoimmune diseases when produced in excess. Many antibodies are created to inhibit over-expressed TNF-alpha but with serious side effects. The present invention provides a mRNA vaccine to reduce the titer of TNF-alpha, leading to treating multiple life-threatening diseases including but not limited to rheumatoid arthritis, psoriatic arthritis, Crohn's disease, ankylosing spondylitis, plaque psoriasis, inflammatory bowel disease, type 2 diabetes, tumor progression, angiogenesis, and metastasis, heart failure from myocardial cell death, and atherosclerosis from fibrosis.

### SEQUENCE LISTING

The instant application contains a Sequence Listing, which has been submitted electronically in ASCII format and is hereby incorporated by reference in its entirety. Said ASCII text file, created on May 24, 2024, is named TNF_SL_2024-908_Sequence Listing, and is 16 kb in size.

### INTRODUCTION

Tumor Necrosis Factor-alpha (TNF-alpha) is a cytokine with a significant role in the immune system, particularly in inflammation and the acute phase response. However, its dysregulation or overexpression can lead to various diseases, primarily through mechanisms of chronic inflammation and immune system dysregulation. In autoimmune diseases such as rheumatoid arthritis, inflammatory bowel disease, and psoriasis, TNF-alpha contributes to ongoing inflammation and tissue damage. It's also implicated in developing insulin resistance in conditions like obesity and type 2 diabetes by interfering with insulin signaling. In the context of cancer, while TNF-alpha can induce tumor cell death, it can also paradoxically promote tumor progression, angiogenesis, and metastasis. Additionally, in cardiovascular diseases, elevated levels of TNF-alpha are linked to heart failure and atherosclerosis, as it can lead to myocardial cell death and fibrosis. This multifaceted role of TNF-alpha in disease pathology highlights its importance as a target for various therapeutic interventions.

TNF-alpha blockers, also known as TNF-inhibitors, are a class of medications that inhibit the action of TNF-alpha. They are used to treat various autoimmune diseases, such as rheumatoid arthritis, psoriatic arthritis, Crohn's disease, ankylosing spondylitis, and plaque psoriasis. By blocking the activity of TNF-alpha, these drugs reduce inflammation and other symptoms of these diseases.

The mechanism of TNF-alpha blockers involves binding to TNF-alpha, preventing it from activating TNF receptors on cell surfaces. This blockade reduces the inflammatory response, beneficial in diseases where inflammation is part of the pathology. However, since TNF-alpha is also involved in normal immune function, using TNF blockers can increase the risk of infections.

There are several types of TNF-alpha blockers, including monoclonal antibodies such as rituximab (which targets CD20-positive B cells, used in conditions like rheumatoid arthritis and certain types of vasculitis), infliximab, adalimumab, and golimumab. Also, soluble TNF receptors, the fusion proteins that act as decoy receptors, bind to TNF-alpha, such as etanercept. They are particularly effective for rheumatoid arthritis, psoriatic arthritis, ankylosing spondylitis, Crohn's disease, and ulcerative colitis.

TNF-alpha blockers have expanded the therapeutic options for patients with various inflammatory and autoimmune diseases. Besides the risk of infections and potential for allergic reactions and cancer, TNF-alpha-blockers can also cause other side effects like pain, swelling, itching, or rash at the injection site, worsening or new onset of heart failure symptoms, and rarely, demyelinating disorders like multiple sclerosis that can be exacerbated or triggered.

These drugs can induce the formation of autoantibodies, a newer class of small-molecule drugs, like tofacitinib and baricitinib, inhibit Janus kinase (JAK) pathways and present alternate choices. Traditional Disease-Modifying Anti-Rheumatic Drugs (DMARDs) include methotrexate, sulfasalazine, and hydroxychloroquine, often used with biologics or as first-line therapy in milder cases.

mRNA technology offers an ideal solution to curb the TNF-alpha by creating antibodies against these proteins, often in a controlled manner, such as by employing the epitopes of TNF-alpha protein.

### BACKGROUND OF THE INVENTION

mRNA VACCINES

The heart of the mRNA is the Coding Sequence, comprising codons, which are nucleotide triplets that dictate the amino acid sequence in the resulting protein. Following the coding sequence is the 3′ Untranslated Region (3′ UTR), which, like its 5′ counterpart, doesn't code for protein but plays a role in mRNA stability and translation regulation. Finally, the Poly-A Tail, a string of adenine nucleotides at the mRNA's 3′ end, further stabilizes the mRNA and influences its lifespan for translation, assisting in its transport from the nucleus to the cytoplasm. Additionally, Ribosome Binding Sites, primarily located within the 5′ UTR, are critical for correct ribosome assembly and translation initiation on the mRNA.

The number of protein molecules generated from a single mRNA is primarily determined by “translation efficiency.” The stability of the mRNA molecule, the availability of different translation components, and the existence of translation initiation sites are some factors affecting translation efficiency.

The length of the mRNA, translation efficiency, and stability of the resultant protein all affect how many protein molecules can be translated from a single mRNA molecule. It is noteworthy that translation is a dynamic process and that a chain of ribosomes known as polysomes can be formed when multiple ribosomes simultaneously translate the same mRNA molecule. Various ribosomes can translate a single mRNA molecule; this phenomenon is known as polysome or ribosome “clustering.” This makes it possible to produce several protein molecules from the same mRNA template effectively and concurrently. Several factors, including ribosome availability, cellular circumstances, and particular mRNAs and their associated regulatory elements, govern how many ribosomes can translate mRNA simultaneously. The process of developing mRNA vaccines comprises several well-defined but complex steps:

Select an Antigenic Epitope: Start by identifying a specific sequence or epitope from a protein that is known to be antigenic. Antigenic epitopes are protein regions the immune system recognizes, typically as part of an antibody-antigen interaction.

Design mRNA Sequence: Design an mRNA sequence that encodes the selected epitope. The mRNA sequence should follow the rules of mRNA transcription, such as starting with a 5′ cap and including a 3′ poly-A tail. Ensure the sequence is in-frame with the ribosome so that translation produces the desired epitope.

Codon Optimization: Optimize the mRNA sequence for translation efficiency in the desired host cell. This may involve choosing more frequently used codons in the host organism to ensure efficient translation.

Consider mRNA Modifications: To enhance stability and translation efficiency, consider incorporating modified nucleotides, such as pseudouridine or 5-methylcytidine, into the mRNA sequence. These modifications can improve mRNA stability and reduce immune recognition. Also significant is the replacement of uridine with pseudouridine.

Delivery Method: Determine how to deliver the mRNA to the target cells. This can include electroporation, lipid nanoparticles, or viral vectors.

Expression System: Choose an appropriate expression system for mRNA, such as a cell line or organism that can efficiently translate the mRNA and produce the epitope.

In Vitro Translation: Transcribe and translate the mRNA in an in vitro translation system, such as a cell-free translation system or using cultured cells. This will help verify that the mRNA is producing the desired epitope.

Antigen Presentation: Once the mRNA has been translated into the antigenic peptide within the target cells, it can be processed and presented on the cell surface by major histocompatibility complex (MHC) molecules. This presentation is essential for immune recognition.

Immunization: Use the translated peptide to immunize animals or individuals to stimulate an immune response. Use this antigenic peptide as part of a vaccine or immunotherapy.

Immune Response Evaluation: Monitor the immune response by measuring the production of antibodies or T-cell responses against the antigenic peptide. Use techniques such as ELISA, flow cytometry, or cytokine assays to assess the immune response.

The complex process of matching a sequence of the Fc region of an antibody to an epitope sequence involves a synergy of bioinformatics and molecular biology techniques. Discerning the distinct roles and structures of the Fc region and epitope is pivotal. The Fc region interacts with cell surface receptors and complement proteins at an epitope, the specific portion of an antigen recognized by the immune system.

The second phase entails retrieving and aligning these sequences, a process facilitated by tools and databases like GenBank and BLAST. When 3D structures are obscure, homology modeling tools become instrumental in predicting these structures. Software such as PyMOL and UCSF Chimera enables researchers to visualize and analyze these structures in detail.

The enigma of the interaction between the Fc region and the epitope begins to unravel during the docking studies. Tools like HADDOCK and ClusPro simulate these intricate interactions, revealing binding affinities and interactive sites. Experimental validation, however, is indispensable. Techniques like site-directed mutagenesis and binding assays, such as ELISA or Surface Plasmon Resonance, are deployed, providing empirical data that corroborate the in-silico findings.

Data interpretation is another crucible where statistical tools play a significant role. They sift through the conglomerate of data, delineating substantial patterns and insights and leading to coherent conclusions. Further in vivo studies augment these findings, offering a comprehensive view of the Fc-epitope interactions within a biological context.

The B epitope linkers are short peptide sequences that connect different B cell epitopes to create chimeric proteins or multi-epitope antigens for various applications, including vaccine development.

mRNA vaccines utilize in vivo ribosomes to express or translate antigens that can create antibodies. To reduce TNF-alpha levels, antibodies can be formed against this cytokine by carefully selecting epitopes from the base of the molecule and its projected isoforms.

An epitope, also known as an antigenic determinant, is a specific part of an antigen recognized and bound by an antibody, B-cell receptor, or T-cell receptor during an immune response. Antigens are substances (often proteins) that can trigger an immune response in the body, and they may be part of a pathogen such as a virus or bacterium or a foreign substance like pollen. Epitopes can be categorized into two main types. The linear or sequential epitopes consist of a linear sequence of amino acids within the antigen's primary structure. They are recognized by their amino acid sequence rather than their three-dimensional structure. The conformational or discontinuous epitopes are formed by amino acids that are not sequential in the primary protein sequence but are brought together in space by protein folding. These epitopes are recognized by their three-dimensional structure. Given any amino acid sequence, the B and T-cell epitopes can be calculated (http://tools.iedb.org).

The interaction between an epitope and an antibody or a receptor is specific; a particular epitope will bind to a specific antibody or receptor with high specificity. This specificity is fundamental to the immune system's ability to detect and respond to various antigens. Understanding epitopes is crucial in multiple fields, including vaccine development, immunotherapy, and diagnostic testing.

Epitopes are the specific regions of antigens (in this case, the autoantibodies) that are recognized by autoantigens. For MHC binding, epitopes must bind to major histocompatibility complex (MHC) molecules to be presented to T cells. For class I MHC, epitopes are typically 8-11 amino acids in length, while for class II MHC, they are usually longer, around 15-24 amino acids. Some epitopes may be discontinuous and composed of amino acids not adjacent to the protein sequence. B cell epitopes are usually 5-17 amino acids in length but can be extended without adverse effects on the immune function.

B cell epitopes are typically fragments located on the outer surface of a (native) protein or peptide antigens, preferably having 8 to 15 amino acids, which may be recognized by antibodies, i.e., in their native form. This invention used a 5-25 amino acid sequence cutoff since epitopes are rarely found beyond these limits. Such epitopes of proteins or peptides may be selected from any of the variants of such proteins or peptides. In this context, antigenic determinants can be conformational or discontinuous epitopes, which are composed of segments of the proteins or peptides as defined herein that are discontinuous in the amino acid sequence of the proteins or peptides as defined herein but are brought together in the three-dimensional structure or continuous or linear epitopes which are composed of a single polypeptide chain. In the context of the present invention, an epitope may be the product of the translation of a provided coding mRNA as specified herein.

The next step is to convert the epitope sequences into nucleoside coding sequences by first converting the target polypeptide sequence into DNA through reverse transcription and then to RNA. First, multiple epitopes from the same protein or its isoforms can be linked together using linkers including but not limited to various combinations of Alanine, Asparagine, Glutamic Acid, Glycine, Leucine, Lysine, Phenylalanine, Proline, Serine, and Threonine, or combinations thereof, using singularly or as repeated groups.

mRNA vaccine functional regulation requires untranslated regions (UTRs) between the open reading frame (ORF) and the 5′ and 3′ ends, upstream and downstream of the mRNA. These UTRs contain regulatory sequences associated with mRNA stability and efficient and correct mRNA translation. They also help recognize mRNA by ribosomes and help in post-transcriptional modification of the mRNA. The mRNA translation and its half-life can be improved by including cis-regulatory sequences in the UTRs. Additionally, the inclusion of naturally occurring sequences, such as those derived from alpha- and beta-globins, have been widely used to design mRNA constructs for vaccines.

mRNA vaccine molecules are large (104-106 Da) and negatively charged. They are unable to pass through the lipid bilayer of cell membranes. Naked mRNA would be destroyed and degraded by the nucleases in the bloodstream. In addition, naked mRNA is also attached and engulfed by immune cells in the tissue and the serum. Methods to deliver mRNA molecules into the cells include gene guns, electroporation, and ex vivo transfection. The in vivo methods of delivering mRNA involve transfection immune or non-immune cells using lipids or transfecting agents.

Although naked mRNA, liposomes, and polyplexes have shown clinical effectiveness in humans, LNPs for mRNA vaccines are the only drug delivery system that has demonstrated clinical efficacy and has been approved for human use. The COVID-19 mRNA vaccines against SARS-COV-2, developed by Moderna and Pfizer/BioNTech, employ LNPs to deliver the mRNA payload to the body. LNPs are currently the foremost non-viral delivery vector employed for gene therapy. The clinical effectiveness of LNPs was first demonstrated when LNP-siRNA therapeutic Onpattro® (patisiran) was approved by the US FDA for hereditary transthyretin-mediated amyloidosis. LNP formulations are the most successful, effective, and safe method of delivery of mRNA vaccines for human immunizations. LNPs offer numerous advantages for mRNA delivery to the site of action, including ease of formulation and scale-up, highly efficient transfection capacity, low toxicity profile, modularity, compactivity with different nucleic acid types and sizes, protection of mRNA from internal degradation, and increasing the half-life of mRNA vaccines. LNPs are typically composed of four components: an ionizable cationic lipid, a helper phospholipid, cholesterol, and a PEGylated lipid. These lipids encapsulate the mRNA vaccine's payload and protect the nucleic acid core from degradation.

### DETAILS OF THE INVENTION

Messenger RNA (mRNA) is crucial in translating genetic information from DNA into proteins. It includes several key components, each with a specific function. The 5′ Cap, a modified guanine nucleotide at the mRNA's 5′ end, ensures mRNA stability and aids in translation initiation, protecting the mRNA from degradation and assisting in ribosome recognition. Adjacent to the cap is the 5′ Untranslated Region (5′ UTR), a sequence not coding for protein but crucial in regulating translation efficiency and ribosome binding.

Advantageously, mRNA can be manufactured in a large-scale fashion and enables the production of a robust immune response based on mRNA encoding, for example, antigens that produce antibodies specific to proteins of the target infecting cell.

In various embodiments, the coding mRNA comprises, preferably in 5′- to 3′-direction, the following elements:

5′-cap structure selected from m7G(5′), m7G(5′)ppp(5′)(2OMeA), or m7G(5′)ppp(5′)(2′OMeG);

The 5′-terminal start element is selected from AUG, GUG, and UUG.

### 3-UTR

An open reading frame to express protein

poly(A) sequence comprising about 50 to about 250 adenosines.

The 5′ end of the mRNA contains a 7-methylguanosine (m7G) moiety, followed by a triphosphate moiety to the first nucleotide (m7GpppN). m7GpppN is called a 5′ cap, a protective structure that protects RNA from exonuclease cleavage, regulates pre-mRNA splicing, and initiates mRNA translation and nuclear export of the mRNA to the cytoplasm. The 5′ cap is also essential in recognizing non-self mRNA or exogenous mRNA from self mRNA or the endogenous mRNA by the innate immune system.

The mRNA can be modified to improve its efficacy and stability by introducing many post-transcriptional modifications. Some of these include 2′-O-methylation at position 2′ of the ribose ring at the first nucleotide (Cap 1, m7GpppN1m) and the second nucleotide e (Cap 2, m7GpppN1mN2m) as well. These modifications in the 5′ cap structure not only increase the translation efficiency of mRNA but also stop the activation of endosomal and cytosolic receptors, including RIG-I and MDA5, which act as defensive mechanisms against viral mRNA.

Hence, the 2′-O-methylation of the 5′ cap structure is a highly desirable property for increasing and enhancing the protein production from the mRNA after its transcription and blocking any undesirable immune responses from the host immune system to the antigenic IVT mRNA. This 5′ cap can be achieved by adding S-adenosyl methionine and the Cap 0 structure to the IVT mRNA reaction, which yields IVT mRNA with the Cap 1 structure and S-adenosyl-L-homocysteine. Cap 1 refers to m7GpppNm, where Nm represents any nucleotide with a 2′O methylation. This structure plays a crucial role in RNA stability and the initiation of protein synthesis. m7G represents a 7-methylguanosine residue. It's a modified guanine nucleotide with a methyl group attached to the nitrogen at the 7th position. This modification is crucial for RNA stability and efficient translation; ppp is a triphosphate bridge. It connects the 5′ end of the mRNA with the m7G cap. This linkage is unusual because it's a 5′-to-5′ triphosphate linkage, unlike the typical 5′-to-3′ phosphodiester bonds in RNA; Am signifies a 2′-O-methyladenosine residue. It's a modification where a methyl group is added to the 2′ hydroxyl group of the first nucleotide of the mRNA adjacent to the cap. This modification can enhance the stability of the mRNA and also plays a role in distinguishing self-RNA (e.g., from a cell's genes) from non-self-RNA (e.g., viruses or other pathogens) in the immune response.

The cap1 structure (m7GpppAm) is a common feature in eukaryotic mRNA and is essential for various aspects of RNA metabolism, including RNA stability, export from the nucleus, and translation initiation. It also helps recognize the mRNA by the ribosome and other components of the translation machinery. In the modified structure, there's an additional methyl group at the 3′ position of the m7G cap (m7G+m3′). This modification might further influence the interaction of the cap with cellular proteins and potentially affect mRNA stability and translation efficiency.

An example of the modified 5′-cap1 structure (m7G+m3′-5′-ppp-5′-Am) can be found in certain messenger RNAs (mRNAs) used in mRNA-based vaccines, such as those developed for COVID-19. In these vaccines, the mRNA carries the instructions to produce a specific viral protein (like the spike protein of the SARS-COV-2 virus) that triggers an immune response in the body. The modified cap structure plays a crucial role in these mRNA molecules.

The various types of CAPs that can be beneficial in the present invention include ARCA, Bridged Cap (BCAP), Cap0, Cap1, Cap2, Cap3, Cap4, CleanCap, Hypermodified Caps, Modified Cap1, Synthetic or Designer Caps, Tobacco Mosaic Virus (TMV) Cap, and

In preferred embodiments, the cap1 structure of the coding mRNA of the invention is formed using co-transcriptional capping using tri-nucleotide cap analogs m7G(5′)ppp(5′)(2′OMeA)pG or m7G(5′)ppp(5′)(2′OMeG)pG. A preferred cap1 analog that may suitably be used in manufacturing the coding mRNA of the invention is m7G(5′)ppp(5′)(2′OMeA)pG.

In the context of mRNA therapeutics, heterologous peptides are often used as signal sequences to direct the synthesized protein to specific locations within the cell, such as the endoplasmic reticulum (ER) or extracellular space. These signal peptides play a crucial role in ensuring that the protein encoded by the mRNA reaches its intended destination to function effectively. Some commonly used heterologous signal peptides in mRNA therapeutics include:

Human Ig kappa chain signal peptide (Igκ): Derived from the immunoglobulin kappa light chain, this signal peptide is widely used to direct the expressed protein to the secretory pathway.

Honeybee melittin signal peptide: Originally from the honeybee venom, the melittin signal sequence is known for its efficiency in directing proteins for secretion.

Tissue plasminogen activator signal peptide (tPA): tPA signal sequence is commonly used to efficiently process proteins. It is derived from the human tissue plasminogen activator.

Granulocyte-macrophage colony-stimulating factor signal peptide (GM-CSF): Taken from the GM-CSF, this signal peptide is used to direct proteins to the extracellular space or the secretory pathway.

Albumin signal peptide: This signal sequence, derived from human serum albumin, is employed to guide proteins towards secretion.

Signal peptide of the human erythropoietin (EPO): The EPO signal peptide is used in certain therapeutic applications where efficient secretion of the protein is desired.

Secretory Pathway Signal Peptide (Sec/SPI): A common signal peptide directing proteins to the secretory pathway.

Preproinsulin Signal Peptide: Derived from preproinsulin, this signal peptide is used for insulin analogs and other therapeutic proteins requiring secretion.

Alpha Factor Signal Sequence (from Saccharomyces cerevisiae): Commonly used in yeast expression systems to direct protein secretion.

Basic Secretory Signal Peptide (BSSP): A synthetic signal peptide designed for efficient protein secretion.

Endothelin Signal Peptide: Used for directing proteins to the secretory pathway, particularly in cardiovascular therapeutics.

Interferon-alpha Signal Peptide: Employed in producing type I interferon and related proteins.

Lysozyme Signal Peptide: Derived from lysozyme, this peptide directs proteins to the secretory pathway in various expression systems.

Calreticulin Signal Sequence: Used for targeting proteins to the endoplasmic reticulum.

Vascular Endothelial Growth Factor (VEGF) Signal Peptide: Used in angiogenesis and tissue regeneration therapies.

Beta-Lactamase Signal Peptide: Often used in bacterial expression systems for protein secretion.

p67 Signal Sequence (from Trypanosoma brucei): Employed in certain therapeutic applications for targeting proteins to specific cellular compartments.

Glucagon Signal Peptide: Used in the expression of glucagon-like peptides and analogs.

The invention's open reading frame or coding sequence of mRNA can be prepared using any method known in the art, including chemical synthesis, such as, e.g. solid phase mRNA synthesis, and in vitro methods, such as mRNA in vitro transcription reactions.

In a preferred embodiment, the coding mRNA, preferably the mRNA, is obtained by mRNA in vitro transcription.

In embodiments, a typical selection of the components of an mRNA vaccine based on TNF-alpha epitopes and peptides selected from P01375·TNFA_HUMAN (UniProt) to create an open reading frame portion of mRNA in mRNA vaccine are given below:

Sequence No. 01 identifies TNF-alpha peptide″ its sequence comprises:
″KGQGCPSTHVLLTHTISRIAVSYQTKVNLLSAIKSPCQRETPEGAEAKPWYEPIYLGGVF
QLEKGDRLSAEINRPDYLDFAES.″
Sequence No. 02 identifies ″TNF-alpha peptide epitope 1,″ and its sequence
comprises:
″IRDVELAEEALPKKTGGPQGS.″
Sequence No. 03 identifies ″TNF-alpha epitope 2″; its sequence comprises:
″IGPQREE.″
Sequence No. 04, another ″TNF-alpha epitope 3,″ has a sequence comprising
″DLSLISPLAQAVRSSSRTPSDKPVA.″
Sequence No. 05 identifies ″TNF-alpha epitope 4,″ and its sequence comprises:
″VANPQAEGQLQWLNRRANAL.″
Sequence No. 06 identifies the ″TNF-alpha epitope 5,″ its sequence comprises
″GQGCPSTHV.″
Sequence No. 07 is a ″TNF-alpha epitope 6,″ Its sequence comprises
″IAVSYQTK.″
Sequence No. 08 identifies ″TNF-alpha epitope 7,″ its sequence comprises:
″SPCQRETPEGAEAKPW.″
Sequence No. 09 identifies ″TNF-alpha epitope 8,″ and its sequence comprises:
″GDRLSAEINRPDYLDFAES.″
″TNF-alpha mRNA vaccine Cap2″ sequence comprises:
″GA.″
Sequence No. 10 identifies ″TNF-alpha mRNA vaccine 5'-UTR,″ its sequence comprises:
″GAATAAACTAGTATTCTTCTGGTCCCCACAGACTCAGAGAGAACCCGCCACC.″
Sequence No. 11 identifies ″TNF-alpha mRNA vaccine Signal Peptide,″ its sequence
comprises:
″ATGTTCGTGTTCCTGGTGCTGCTGCCTCTGGTGTCCAGCCAGTGTGTG.″
Sequence No. 12, identifies ″TNF-alpha mRNA vaccine 3'UTR,″ its sequence comprises:
″GCTAGCTGCCCCTTTCCCGTCCTGGGTACCCCGAGTCTCCCCCGACCTCGGGTCCCA
GGTATGCTCCCAC-CTCCACCTGCCCCACTCACCAC-
CTCTGCTAGTTCCAGACACCTCCCAAGCACGCAG-CAATGCAGCTCAAAAC-
GCTTAGCCTAGCCACACCCCCACGGGAAACAG-
CAGTGATTAACCTTTAGCAATAAACGAAA-GTTTAACTAA-
GCTATACTAACCCCAGGGTTGGTCAATTTCGTGCCAGCCACACCCTGGAGCTAGC.″
Sequence No. 13 identifies ″TNF-alpha mRNA vaccine Poly(A),″ its sequence comprises:
″AAAAAAAAAAAAAAAAAAAAAAAAAAAAAA-
GCATATGACTAAAAAAAAAAAAAAAAAAAAAAAAAAAAAAAAAAAAAAAAAAAAA
AAAAAAAAAAAAAAAAAAAAAAAAA.″
Sequence14 identifies ″Amino acid sequence combining epitopes and peptide of TNF-
alpha vaccine expression comprises:
AVSYQTKDLSLISPLAQAVRSSSRTPSDKGDRLSAEINRPGQGCPSTHVIAVSYQTKIGPQR
EEDHLLEPRINRPDYLDFAESIRDVELAEEALPKKTGGPQGSNRPDYLDFAESQAEGQLQ
WLNRRANALSPCQRETPEGAEAKPWVANPQAEGVRSSSRTPSDKPVAYLDFAKGQGCPS
THVLLTHTISRIAVSYQTKVNLLSAIKSPCQRETPEGAEAKPWYEPIYLGGVFQLEKGDRL
SAEINRPDYLDFAES.
Sequence No. 15 identifies ″Amino acid sequence combining epitopes and peptide of
TNF-alpha vaccine expression with linkers;″ its sequence comprises
AVSYQTKGSGSGSGSDLSLISPLAQAVRSSSRTPSDKGSGSGSGSGDRLSAEINRPGSGSG
SGSGQGCPSTHVGSGSGSGSIAVSYQTKGSGSGSGSIGPQREEDHLLEPRGSGSGSGSINR
PDYLDFAESGSGSGSGSIRDVELAEEALPKKTGGPQGSGSGSGSGSNRPDYLDFAESGSG
SGSGSQAEGQLQWLNRRANALGSGSGSGSSPCQRETPEGAEAKPWGSGSGSGSVANPQ
AEGGSGSGSGSVRSSSRTPSDKPVAGSGSGSGSYLDFAGSGSGSGSKGQGCPSTHVLLTH
TISRIAVSYQTKVNLLSAIKSPCQRETPEGAEAKPWYEPIYLGGVFQLEKGDRLSAEINRP
DYLDFAES.″
Sequence No. 16 identifies ″Open Reading Frame to express epitopes and peptide of
TNF-alpha in mRNA vaccine″, it comprises (replacing U with T for ST.26 compliance):
GCGGTGAGCTATCAGACCAAAGGCAGCGGCAGCGGCAGCGGCAGCGATCTGAGCCT
GATTAGCCCGCTGGCGCAGGCGGTGCGCAGCAGCAGCCGCACCCCGAGCGATAAAG
GCAGCGGCAGCGGCAGCGGCAGCGGCGATCGCCTGAGCGCGGAAATTAACCGCCCG
GGCAGCGGCAGCGGCAGCGGCAGCGGCCAGGGCTGCCCGAGCACCCATGTGGGCAG
CGGCAGCGGCAGCGGCAGCATTGCGGTGAGCTATCAGACCAAAGGCAGCGGCAGCG
GCAGCGGCAGCATTGGCCCGCAGCGCGAAGAAGATCATCTGCTGGAACCGCGCGGC
AGCGGCAGCGGCAGCGGCAGCATTAACCGCCCGGATTATCTGGATTTTGCGGAAAGC
GGCAGCGGCAGCGGCAGCGGCAGCATTCGCGATGTGGAACTGGCGGAAGAAGCGCT
GCCGAAAAAAACCGGCGGCCCGCAGGGCAGCGGCAGCGGCAGCGGCAGCGGCAGC
AACCGCCCGGATTATCTGGATTTTGCGGAAAGCGGCAGCGGCAGCGGCAGCGGCAGC
CAGGCGGAAGGCCAGCTGCAGTGGCTGAACCGCCGCGCGAACGCGCTGGGCAGCG
GCAGCGGCAGCGGCAGCAGCCCGTGCCAGCGCGAAACCCCGGAAGGCGCGGAAGC
GAAACCGTGGGGCAGCGGCAGCGGCAGCGGCAGCGTGGCGAACCCGCAGGCGGAA
GGCGGCAGCGGCAGCGGCAGCGGCAGCGTGCGCAGCAGCAGCCGCACCCCGAGCG
ATAAACCGGTGGCGGGCAGCGGCAGCGGCAGCGGCAGCTATCTGGATTTTGCGGGCA
GCGGCAGCGGCAGCGGCAGCAAAGGCCAGGGCTGCCCGAGCACCCATGTGCTGCTG
ACCCATACCATTAGCCGCATTGCGGTGAGCTATCAGACCAAAGTGAACCTGCTGAGC
GCGATTAAAAGCCCGTGCCAGCGCGAAACCCCGGAAGGCGCGGAAGCGAAACCGTG
GTATGAACCGATTTATCTGGGCGGCGTGTTTCAGCTGGAAAAAGGCGATCGCCTGAG
CGCGGAAATTAACCGCCCGGATTATCTGGATTTTGCGGAAAGC

TABLE 1
mRNA Vaccine design

| Element | Description |
|---|---|
| Cap | A modified 5′-cap1 structure (m7G + m3′-5′-ppp-5′-Am) GA |
| 5′-UTR | The 5′-untranslated region derived from human alpha-globin |
| | RNA with an optimized Kozak sequence. (Sequence No. 10) |
| Signal | S glycoprotein signal peptide (extended leader sequence) |
| peptide | guides translocation of the nascent polypeptide chain |
| | into the endoplasmic reticulum (Sequence No. 11) |
| Coding | Codon-optimized sequence: Sequence No. 01-Sequence |
| region | No. 9, individually, or a combination thereof. |
| 3′-UTR | The 3′ untranslated region comprises two sequence |
| | elements derived from the amino-terminal enhancer of |
| | split (AES) mRNA and the mitochondrial encoded 12S |
| | ribosomal RNA to confer RNA stability and high total |
| | protein expression: (Sequience No. 12) |
| poly(A) | A 110-nucleotide poly(A)-tail consisting of a stretch of |
| | 30 adenosine residues, followed by a 10-nucleotide |
| | linker sequence and another 70 adenosine residues: |
| | (Sequence No. 13) |

In embodiments, the nucleotide mixture used in mRNA in vitro transcription may additionally contain modified nucleotides as defined herein. Modifying codons, particularly in codon optimization, involves various strategies to enhance gene expression or protein synthesis in a target organism. In codon optimization, structural changes to the RNA sequence are often made to enhance the stability and efficiency of mRNA translation. These changes are designed to avoid hindrances in the translation process and improve overall protein expression.

Altering sequences that form stable hairpin or stem-loop structures in the mRNA. For instance, a sequence like GGGGGG, which might form a strong secondary structure, could be altered to a less self-complementary sequence like GAGAGA without changing the amino acid sequence.

Modifying the GC content of the mRNA to optimize stability and efficiency. High GC content can lead to secondary solid structures, while low GC content might reduce mRNA stability. Adjustments are made to reach an optimal balance. For example, replacing AT-rich codons with GC-rich synonymous codons or vice versa.

Removing or altering sequences that are known to signal for rapid mRNA degradation. For example, specific sequences like AU-rich elements in eukaryotes might be modified to increase the half-life of the mRNA.

Changing sequences can cause the ribosome to stall during translation. For instance, a stretch of rare codons or a sequence that forms a tight secondary structure might be modified to ensure smooth progression of the ribosome.

Altering sequences that could mimic regulatory elements like promoters, enhancers, or internal ribosome entry sites (IRES) which could interfere with proper transcription and translation.

Adjusting codon usage to match the tRNA pool of the host organism. Overusing a particular codon can deplete its corresponding tRNA, slowing translation. The sequence is modified to use more abundant tRNAs.

Reducing repetitive sequences that can lead to recombination events or genomic instability. This also helps in avoiding slippage during transcription or translation.

These structural changes are tailored to the specific requirements of the host organism and the protein being expressed. The goal is to create an mRNA sequence that is efficiently translated with minimal interruptions or instability, leading to higher protein yields.

In that context, preferred modified nucleotides comprise pseudouridine, N1-methylpseudouridine, 5-methylcytosine, and 5-methoxyuridine. Embodiments of uracil nucleotides in the nucleotide mixture are replaced (either wholly or partially) by pseudouridine and/or N1-methyl pseudouridine to obtain a modified coding mRNA.

In preferred embodiments, the nucleotide mixture (i.e., the fraction of each nucleotide in the mix) used for mRNA in vitro transcription reactions may be optimized for the given mRNA sequence.

In a further preferred embodiment, the coding mRNA, particularly the purified coding mRNA, is lyophilized. The mRNA of the invention, particularly the purified mRNA, may also be dried using spray-drying or spray-freeze drying.

A second aspect relates to a vaccine comprising at least one coding mRNA of the first aspect.

Notably, embodiments relating to the vaccine of the second aspect may likewise be read on and be understood as suitable embodiments of the vaccine of the third aspect. Also, embodiments relating to the vaccine of the third aspect may likewise be read on and be understood as suitable embodiments of the vaccine of the second aspect (comprising the mRNA of the first aspect). In preferred embodiments of the second aspect, said vaccine comprises at least one mRNA encoding peptides or proteins according to the first aspect, or an immunogenic fragment or immunogenic variant thereof, wherein said vaccine is to be, preferably, administered intramuscularly or intradermal.

Preferably, intramuscular or intradermal administration of the said vaccine results in the expression of the encoded antigen in a subject. Preferably, the vaccine of the second aspect is suitable for an ideal vaccine.

The vaccine may comprise a safe and effective amount of the mRNA to result in the encoded antigenic protein's expression and activity. At the same time, a “safe and effective amount” is small enough to avoid serious side effects.

In the context of the invention, a “vaccine” refers to any type of vaccine in which the specified ingredients (e.g., mRNA encoding proteins or peptides, e.g., in association with a polymeric carrier or LNP), may be incorporated, optionally along with any further constituents, usually with at least one pharmaceutically acceptable carrier or excipient. The vaccine may be a dry vaccine, such as a powder or granules, or a solid unit, such as a lyophilized form. Alternatively, the vaccine may be liquid, and each constituent may be independently incorporated in dissolved or dispersed (e.g., suspended or emulsified) form.

In a preferred embodiment of the second aspect, the vaccine comprises mRNA coding at least one protein or peptide and, optionally, at least one pharmaceutically acceptable carrier or excipient.

In particularly preferred embodiments of the second aspect, the vaccine comprises at least one coding mRNA, wherein the coding mRNA includes or consists of an mRNA sequence that is identical or at least 70% to 99% to a nucleic acid sequence selected from the group consisting of epitopes chosen, and, optionally, at least one pharmaceutically acceptable carrier or excipient.

The term “pharmaceutically acceptable carrier” or “pharmaceutically acceptable excipient” as used herein preferably includes the liquid or non-liquid basis of the vaccine for administration. If the vaccine is liquid, the carrier may be water, e.g., pyrogen-free water; isotonic saline or buffered (aqueous) solutions, e.g., phosphate, citrate, etc. buffered solutions.

Furthermore, organic anions of the cations may be in the buffer. Accordingly, in embodiments, the mRNA vaccine of the invention may comprise pharmaceutically acceptable carriers or excipients using one or more pharmaceutically acceptable carriers or excipients to, e.g., increase stability, increase cell transfection, permit the sustained or delayed, increase the translation of encoded epitopes. In addition to traditional excipients such as all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with polynucleotides, hyaluronidase, nanoparticle mimics and combinations thereof. In embodiments, one or more compatible solid or liquid fillers, diluents, or encapsulating compounds, which are suitable for administration to a subject, may also be used.

The term “compatible,” as used herein, means that the constituents of the vaccine are capable of being mixed with mRNA and, optionally, a plurality of mRNAs of the vaccine in such a manner that no interaction occurs, which would substantially reduce the biological activity or the pharmaceutical effectiveness of the vaccine under typical use conditions (e.g., intramuscular, or intradermal administration).

At least one pharmaceutically acceptable carrier or excipient of the vaccine may preferably be selected to be suitable for intramuscular or intradermal delivery. Accordingly, the vaccine is preferably a pharmaceutical vaccine suitable for intramuscular or intradermal administration.

The pharmaceutical vaccine is contemplated for use, but is not limited to, humans and other primates; mammals, including commercially relevant mammals such as cattle, pigs, horses, sheep, cats, dogs, mice, and rats; and birds, including commercially relevant birds such as poultry, chickens, ducks, geese, and turkeys.

Pharmaceutical vaccines of the present invention may suitably be sterile and pyrogen-free. Furthermore, one or more compatible solid or liquid filler diluents or encapsulating compounds, which are suitable for administration to a person, may also be used. The term “compatible,” as used herein, means that the constituents of the vaccine are capable of being mixed with mRNA and, optionally, the further coding mRNA of the vaccine in such a manner that no interaction occurs, which would substantially reduce the biological activity or the pharmaceutical effectiveness of the vaccine under typical use conditions.

In embodiments, the vaccine, as defined herein, may comprise a plurality of or at least more than one of the coding mRNA species as defined in the context of the first aspect of the invention.

In a preferred embodiment of the second aspect, the coding mRNA is complexed or associated with or at least partially complexed or partially associated with one or more cationic or polycationic compounds, preferably cationic or polycationic polymer, cationic or polycationic polysaccharide, cationic or polycationic lipid, cationic or polycationic protein, cationic or polycationic peptide, or any combinations thereof.

Coding mRNA is complexed with protamine in a preferred embodiment of the second aspect. In this context, it is particularly preferred that coding mRNA is complexed or at least partially complexed with a cationic or polycationic compound and a polymeric carrier, preferably cationic proteins or peptides.

In a preferred embodiment of the second aspect, coding mRNA is complexed or partially complexed, with at least one cationic or polycationic protein, peptide, or a combination thereof.

According to embodiments, the vaccine of the present invention comprises the coding mRNA as defined in the context of the first aspect and a polymeric carrier.

A suitable polymeric carrier may be a polymeric carrier formed by disulfide-crosslinked cationic compounds. The disulfide-crosslinked cationic compounds may be the same or different from each other. The polymeric carrier can also contain further components. The polymeric carrier used in the present invention may comprise mixtures of cationic peptides, proteins, polymers, and optionally further components as defined herein, which are crosslinked by disulfide bonds (via —SH groups).

In a particularly preferred embodiment, the polymeric carrier is a peptide polymer, preferably a polyethylene glycol/peptide polymer as defined above, and a lipid component, preferably a lipidoid component. In a preferred embodiment of the second aspect, coding mRNA of the first aspect is complexed or associated with a polymeric carrier, preferably with a polyethylene glycol/peptide polymer as defined above, and a lipidoid component, wherein the lipidoid component is a compound.

In preferred embodiments of the second aspect, coding mRNA is complexed, encapsulated, partially encapsulated, or associated with one or more lipids (e.g., cationic lipids and/or neutral lipids), thereby forming liposomes, lipid nanoparticles (LNPs), lipoplexes, and nanoliposomes.

The liposomes, lipid nanoparticles (LNPs), lipoplexes, and nanoliposomes with incorporated mRNA may be entirely or partially located in the interior space of the liposomes, lipid nanoparticles (LNPs), lipoplexes, and nanoliposomes, within the membrane, or associated with the exterior surface of the membrane. Incorporating nucleic acid into liposomes is also referred to herein as “encapsulation,” wherein the mRNA is entirely contained within the interior space of the liposomes, lipid nanoparticles (LNPs), lipoplexes, and nanoliposomes. The purpose of incorporating an mRNA into liposomes, lipid nanoparticles (LNPs), lipoplexes, and nanoliposomes is to protect the mRNA from an environment that may contain enzymes or chemicals that degrade mRNA and systems or receptors that cause the rapid excretion of the mRNA. Moreover, incorporating an mRNA into liposomes, lipid nanoparticles (LNPs), lipoplexes, and nanoliposomes may promote the uptake of the mRNA and, hence, enhance the therapeutic effect of the mRNA-encoding antigenic peptides. Accordingly, incorporating an mRNA into liposomes, lipid nanoparticles (LNPs), lipoplexes, and nanoliposomes may be particularly suitable for a vaccine, e.g., intramuscular or intradermal administration.

In this context, “complexed” or “associated” refers to the essentially stable combination of coding mRNA of the first aspect with one or more lipids into larger complexes or assemblies without covalent binding.

According to further embodiments, the vaccine of the second aspect may comprise at least one adjuvant. Suitably, the adjuvant is preferably added to enhance the immunostimulatory properties of the vaccine.

The vaccine of the second aspect may comprise, besides the components specified herein, at least one further component which may be selected from the group consisting of further antigens (e.g., in the form of a peptide or protein) or further antigen-encoding nucleic acids, a further immunotherapeutic agent; one or more auxiliary substances (cytokines, such as monokines, lymphokines, interleukins or chemokines); or any further compound, which is known to be immune stimulating due to its binding affinity (as ligands) to human Toll-like receptors; and an adjuvant nucleic acid, preferably an immunostimulatory RNA (isRNA), e.g., CpG-RNA etc.

The LNP formulation is also an adjuvant.

Thirdly, the invention provides a TNF-alpha-inhibiting antibody vaccine. The vaccine comprises the coding RNA of the first aspect and, optionally, the vaccine of the second.

Notably, embodiments relating to the vaccine of the second aspect may likewise be read on and be understood as suitable embodiments of the vaccine of the third aspect. Also, embodiments relating to the vaccine of the third aspect may likewise be read on and be understood as suitable embodiments of the vaccine of the second aspect (comprising the RNA of the first aspect).

In preferred embodiments of the third aspect, the vaccine comprising the first aspect's mRNA or the second aspect's vaccine elicits an adaptive immune response, preferably an adaptive immune response against TNF-alpha proteins known to cause the disease.

In preferred embodiments of the third aspect, the vaccine comprising the first aspect's RNA or the second aspect's vaccine induces strong humoral and cellular immune responses, both B-cell and preferably strong CD4+ and CD8+ T-cell responses.

According to a preferred embodiment of the third aspect, the vaccine, as defined herein, may further comprise a pharmaceutically acceptable carrier and optionally at least one adjuvant as specified in the context of the second aspect.

The vaccine can be used according to the invention for human medical and veterinary medical purposes (mammals, vertebrates, avian species), as a pharmaceutical or vaccine.

Further, the present invention relates to the first medical use of the coding RNA of the first aspect, the second aspect vaccine, and the third vaccine.

Accordingly, the RNA of the first aspect, the vaccine of the second aspect, and the vaccine of the third aspect are used as a medicament.

The present invention provides several applications and uses of the coding RNA of the first aspect, the vaccine of the second, or the vaccine of the third.

RNA vaccine, as a vaccine, may be used for human medical and veterinary medical purposes, preferably for human medical purposes.

In embodiments, the RNA of the first aspect, the vaccine of the second aspect, and the vaccine of the third aspect are used to treat or prophylaxis cytokine effects of TNF-alpha or a disorder related to this cytokine.

The vaccine or the vaccine as defined herein may preferably be administered locally. An intradermal, subcutaneous, intranasal, or intramuscular route may administer vaccines or vaccines. Inventive vaccines or vaccines of the invention are, therefore, preferably formulated in liquid (or sometimes solid) form. In embodiments, conventional needle or needle-free jet injection may administer the inventive vaccine. Preferred in that context is the RNA, the vaccine, and the vaccine administered by intramuscular needle injection.

An mRNA sequence for an antibody against TNF-alpha is produced by selected epitopes and/or peptides, wherein the epitopes and peptides can be optionally linked as a single chain.

1. A coding mRNA vaccine to elicit an adaptive immune response comprising
a) at least one 5′-cap structure;
b) at least one heterologous 5′ untranslated region (5′-UTR);
c) at least one heterologous 3′ untranslated region (3′-UTR);
d) at least one signal peptide;
e) at least one poly(A) tail;
e) at least one coding open reading frame is required to express at least one epitope or a peptide of TNF-alpha protein linked operably to the 3′-UTR and the 5′-UTR.

2. The coding mRNA vaccine of claim 1, wherein the mRNA comprises a poly(A) sequence, preferably comprising 30 to 150 adenosine nucleotides.

3. The coding mRNA vaccine of claim 1, wherein the heterologous signal peptide comprises Sequence No. 11.

4. The coding mRNA of claim 1, where the selected epitope or peptide comprises Sequence No. 01, Sequence No. 02, Sequence No. 03, Sequence No. 04, Sequence No. 05, Sequence No. 06, Sequence No. 07, Sequence No. 08, and Sequence No. 09, or a combination thereof.

5. The coding mRNA of claim 4, wherein the epitopes are combines comprising SEQ. NO. 14 and SEQ NO. 15 epitopes are combined with linkers.

6. The coding mRNA vaccine of claim 1, wherein the epitopes are linked by a linker group comprising Alanine, Asparagine, Glutamic Acid, Glycine, Leucine, Lysine, Phenylalanine, Proline, Serine, and Threonine, or a combination thereof, singularly or in multiple additions, and preferably Glycine-Serine-Glycine-Serine-Glycine-Serine-Glycine-Serine.

7. The coding mRNA vaccine of claim 1, wherein the Open Reading Frame to block TNF-alpha comprises Sequence No. 16.

8. The coding mRNA vaccine of claim 1, wherein RNA sequences are codon-optimized by replacing uridine (U) either wholly or partially by pseudouridine and N1-methyl pseudouridine (T), or a combination thereof.

9. The coding mRNA vaccine of claim 1, wherein the mRNA molecule additionally contains a functional group capable of enhancing the immune response of the mRNA molecule.

10. The coding mRNA vaccine of claim 1, wherein the coding mRNA is a self-replicating RNA, a circular RNA, or a replicon RNA.

11. The coding mRNA vaccine of claim 1, wherein the vaccine optionally comprises at least one pharmaceutically acceptable carrier.

12. The coding mRNA vaccine of claim 1, wherein the coding mRNA is complexed or associated with or at least partially complexed or partially associated with one or more cationic or polycationic compounds, preferably cationic or polycationic polymer, cationic or polycationic polysaccharide, cationic or polycationic lipid, cationic or polycationic protein, cationic or polycationic peptide, or any combinations thereof.

13. The coding mRNA vaccine of claim 1, wherein the coding mRNA is complexed or associated with one or more lipids, thereby forming liposomes, lipid nanoparticles (LNP), lipoplexes, and nanoliposomes, presented with or without lyophilization.

14. The coding mRNA vaccine of claim 12, wherein the LNP essentially consists of
(i) at least one cationic lipid;
(ii) at least one neutral lipid;
(iii) at least one steroid or steroid analog; and
(iv) at least once a PEG-lipid.

15. A method of treating or preventing diseases caused by TNF-alpha cytokine, comprising administering to a human subject needing a coding mRNA vaccine as defined in any of the above claims.

16. The method of claim 15, wherein the diseases comprise rheumatoid arthritis, psoriatic arthritis, Crohn's disease, ankylosing spondylitis, plaque psoriasis, inflammatory bowel disease, type 2 diabetes, tumor progression, angiogenesis and metastasis, heart failure from myocardial cell death, and atherosclerosis from fibrosis, or a combination thereof.


SEQUENCE LISTING (16 sequences)

SEQ ID NO: 1 - protein, 83 aa, Synthetic construct
KGQGCPSTHVLLTHTISRIAVSYQTKVNLLSAIKSPCQRETPEGAEAKPWYEPIYLGGVFQLEKGDRLSAEINRPDYLDFAES

SEQ ID NO: 2 - protein, 21 aa, Homo sapiens
IRDVELAEEALPKKTGGPQGS

SEQ ID NO: 3 - protein, 7 aa, Homo sapiens
IGPQREE

SEQ ID NO: 4 - protein, 25 aa, Homo sapiens
DLSLISPLAQAVRSSSRTPSDKPVA

SEQ ID NO: 5 - protein, 20 aa, Homo sapiens
VANPQAEGQLQWLNRRANAL

SEQ ID NO: 6 - protein, 9 aa, Homo sapiens
GQGCPSTHV

SEQ ID NO: 7 - protein, 8 aa, Homo sapiens
IAVSYQTK

SEQ ID NO: 8 - protein, 16 aa, Homo sapiens
SPCQRETPEGAEAKPW

SEQ ID NO: 9 - protein, 19 aa, Homo sapiens
GDRLSAEINRPDYLDFAES

SEQ ID NO: 10 - protein, 52 aa, Homo sapiens
GAATAAACTAGTATTCTTCTGGTCCCCACAGACTCAGAGAGAACCCGCCACC

SEQ ID NO: 11 - protein, 48 aa, Homo sapiens
ATGTTCGTGTTCCTGGTGCTGCTGCCTCTGGTGTCCAGCCAGTGTGTG

SEQ ID NO: 12 - RNA, 268 nt, Synthetic construct
  source 1..268: mol_type = other RNA
gctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 13 - RNA, 110 nt, Synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 14 - protein, 257 aa, Homo sapiens
AVSYQTKDLSLISPLAQAVRSSSRTPSDKGDRLSAEINRPGQGCPSTHVIAVSYQTKIGPQREEDHLLEPRINRPDYLDFAESIRDVELAEEALPKKTGGPQGSNRPDYLDFAESQAEGQLQWLNRRANALSPCQRETPEGAEAKPWVANPQAEGVRSSSRTPSDKPVAYLDFAKGQGCPSTHVLLTHTISRIAVSYQTKVNLLSAIKSPCQRETPEGAEAKPWYEPIYLGGVFQLEKGDRLSAEINRPDYLDFAES

SEQ ID NO: 15 - protein, 369 aa, synthetic construct
AVSYQTKGSGSGSGSDLSLISPLAQAVRSSSRTPSDKGSGSGSGSGDRLSAEINRPGSGSGSGSGQGCPSTHVGSGSGSGSIAVSYQTKGSGSGSGSIGPQREEDHLLEPRGSGSGSGSINRPDYLDFAESGSGSGSGSIRDVELAEEALPKKTGGPQGSGSGSGSGSNRPDYLDFAESGSGSGSGSQAEGQLQWLNRRANALGSGSGSGSSPCQRETPEGAEAKPWGSGSGSGSVANPQAEGGSGSGSGSVRSSSRTPSDKPVAGSGSGSGSYLDFAGSGSGSGSKGQGCPSTHVLLTHTISRIAVSYQTKVNLLSAIKSPCQRETPEGAEAKPWYEPIYLGGVFQLEKGDRLSAEINRPDYLDFAES

SEQ ID NO: 16 - RNA, 1107 nt, Synthetic construct
  source 1..1107: mol_type = other RNA
gcggtgagctatcagaccaaaggcagcggcagcggcagcggcagcgatctgagcctgattagcccgctggcgcaggcggtgcgcagcagcagccgcaccccgagcgataaaggcagcggcagcggcagcggcagcggcgatcgcctgagcgcggaaattaaccgcccgggcagcggcagcggcagcggcagcggccagggctgcccgagcacccatgtgggcagcggcagcggcagcggcagcattgcggtgagctatcagaccaaaggcagcggcagcggcagcggcagcattggcccgcagcgcgaagaagatcatctgctggaaccgcgcggcagcggcagcggcagcggcagcattaaccgcccggattatctggattttgcggaaagcggcagcggcagcggcagcggcagcattcgcgatgtggaactggcggaagaagcgctgccgaaaaaaaccggcggcccgcagggcagcggcagcggcagcggcagcggcagcaaccgcccggattatctggattttgcggaaagcggcagcggcagcggcagcggcagccaggcggaaggccagctgcagtggctgaaccgccgcgcgaacgcgctgggcagcggcagcggcagcggcagcagcccgtgccagcgcgaaaccccggaaggcgcggaagcgaaaccgtggggcagcggcagcggcagcggcagcgtggcgaacccgcaggcggaaggcggcagcggcagcggcagcggcagcgtgcgcagcagcagccgcaccccgagcgataaaccggtggcgggcagcggcagcggcagcggcagctatctggattttgcgggcagcggcagcggcagcggcagcaaaggccagggctgcccgagcacccatgtgctgctgacccataccattagccgcattgcggtgagctatcagaccaaagtgaacctgctgagcgcgattaaaagcccgtgccagcgcgaaaccccggaaggcgcggaagcgaaaccgtggtatgaaccgatttatctgggcggcgtgtttcagctggaaaaaggcgatcgcctgagcgcggaaattaaccgcccggattatctggattttgcggaaagc